(19) United States
(12) Patent Application Publication
(10) Pub. No.: US 2004/0062738 A1
(43) Pub. Date: Apr. 1, 2004

(54) Products with selected quaternary amino functional silicone elastomers

(75) Inventors: Suman Chopra, Dayton, NJ (US); Amrit Patel, Dayton, NJ (US); Lin Fei, Kendall Park, NJ (US); Saurabh Desai, New Brunswick, NJ (US)

Correspondence Address: Colgate-Palmolive Company, 909 River Road, P.O. Box 1343, Piscataway, NJ, 08855-1343, US

(73) Assignee: Colgate-Palmolive Company

(21) Appl. No.: 10/620,167

(22) Filed: Jul. 15, 2003

Related U.S. Application Data
(60) Provisional application No. 60/399,849, filed on Jul. 31, 2002.

(51) Int. Cl. A61K 7/06; A61K 7/11

(52) U.S. Cl. 424/70.12

(57) ABSTRACT

Personal care products are described which comprise a quaternary amino functional silicone elastomer.

### FIELD OF THE INVENTION

This invention relates to personal care products made with a new type of elastomer.

### BACKGROUND OF THE INVENTION

There is a substantial body of literature describing various types of elastomers and their uses in cosmetic products such as face creams, make-up and antiperspirants/deodorants.

U.S. Pat. No. 4,980,167 to Dow Corning Toray Silicone Company, Ltd.

Describes a cosmetic composition with excellent lubricity made with silicone rubber powder containing 0.50-80 wt % of silicone oil. A smooth and moist texture is obtained.

U.S. Pat. No. 5,599,533 to Estee Lauder Inc. describes a stable water-in-oil emulsion system formed of an organopolysiloxane elastomer, a vehicle in which the elastomer is dispersed or dispersible, a stabilizing agent, a surfactant and an aqueous component and a process for forming the stable water-in-oil emulsion. The stabilized water-in-oil emulsion is useful in personal care products.

U.S. Pat. No. 5,760,116 to General Electric Co. describes elastomer gels containing volatile, low molecular weight silicones and comprising the hydrosilylation addition product of a linear alkenyl functionalized polyorganosiloxane and an MH Q resin and a low molecular weight silicone when subjected to shearing provides for components in personal care formulations that have improved spreadability and substance.

U.S. Pat. No. 5,833,973 to Chesebrough Pond's USA teaches a skin treatment composition is provided which includes a crosslinked non-emulsifying siloxane elastomer, a volatile siloxane and at least 50% by weight of water. Inclusion of the elastomer provides a unique liquid/powdery feel when rubbed into the skin.

U.S. Pat. No. 5,849,314 to Chesebrough-Pond's USA teaches a skin treatment composition comprising a crosslinked non-emulsifying siloxane elastomer, a skin conditioning agent, a volatile siloxane and no more than 5% water. Hydrocarbons such as petrolatum and polyols such as glycerin are the preferred skin conditioning agents. Inclusion of the elastomer provides a non-traditional smooth/silky feel to the skin upon application with a non-draggy rub in.

U.S. Pat. No. 5,929,162 to General Electric Co. teaches a composition comprising the hydrosilylation addition product of a linear alkenyl functionalized polyorganosiloxane and an M^{H}Q resin and a low molecular weight silicone subjected to a new particle size reduction process that includes flow induced shearing and provides for components in personal care formulations that have improved spreadability and substance as a consequence of a unique particle size distribution.

U.S. Pat. No. 5,922,308 to Chesebrough-Pond's USA describes an underarm treatment composition including a deodorant and/or antiperspirant active, a crosslinked non-emulsifying siloxane elastomer and a volatile siloxane. The compositions were found not to undergo syneresis. They also have improved efficacy, faster absorption, anti-whitening and other properties.

U.S. Pat. No. 5,942,215 to Helene Curtis Inc describes an antiperspirant stick composition suitable for topical application to human skin, comprising: an effective amount of an antiperspirant astringent; a volatile silicone; a structurant; and a cross-linked or partially cross-linked non-emulsifying siloxane elastomer.

U.S. Pat. No. 6,060,546 to General Electric Co. describes a non-aqueous silicone emulsion containing a silicone phase and an organic phase which is useful as a component in various personal care compositions. The silicone phase contains a crosslinked silicone elastomer and a low molecular weight silicone fluid and the organic phase contains an organic liquid.

It is an object of the present invention to provide a variety of cosmetic applications including, but not limited to, hair care products, underarm products, and cosmetic creams and lotions made with a new type of elastomer which provides better skin feel. The improvement to such products by the use of these elastomers is to provide improved skin substantivity for silicones which improves moisturization and/or conditioning.

### BRIEF SUMMARY OF THE INVENTION

This invention comprises personal care products such as antiperspirants and/or deodorants; hair care products such as shampoos, hair conditioners, 2-in-1 shampoo/conditioner products, hair styling aids (sprays, gels, mousses); body lotions; make-up compositions (moisturizers, foundations, face creams); liquid soaps; bars; and shower gels. Such personal care products may be improved or restructured by the use of certain quaternary silicone elastomers as described herein.

For purposes of this invention, the quaternary amino functional silicone elastomer (especially in paste form) useful in this invention is of the type prepared by a crosslinking reaction among (1) a partially reduced organohydrogen polysiloxane copolymer of Formula III, below; and one or both of (2) an alkenyl functional silicone gum of Formula IV, below; and (3) an alkenyl terminated polysiloxane of Formula V, below, all in the presence of a volatile and/or non-volatile silicone fluid and a platinum catalyst. Some of these reactions may be found in the art. This initial reaction is followed by a reaction with an alkoxy terminated quaternary amino polysiloxane of Formula VII. The cross-linked silicone gel is swollen in volatile and/or non-volatile silicone fluid. It can be further subjected to high shear force in order to obtain a silicone paste. Since the quaternized silicone elastomer is made in some type of vehicle, it may be prepared containing 50-98 weight % (particularly 70-98%) of volatile and/or non-volatile silicone fluid to give a product with a wide range of viscosities such as in the range of 10,000-500,000 (for example, 50,000-500,000) centipoise (“cps”) as measured by using Brookfield viscometer, LV or RV models. This elastomer paste can be further diluted to a concentration of 2-25 weight % in an emulsion or suspension as desired for use in specific formulations. Personal care products can be formulated by combining the emulsified silicone elastomers of this invention with other ingredients.

In formulating products that are emulsions, an aqueous solution may be added to the material of Formula I in combination with cyclomethicone, hydrocarbons and surfactants. A stable emulsion of silicone elastomer can thus be obtained with a wide range of viscosities such as in the range of 50,000-500,000 cps (for example, 50,000-200,000). Personal care products can be formulated by the addition of different ingredients in either the aqueous phase or the organic phase.

In formulating products that are suspensions, the material of Formula I may be used as a thickening agent to prevent precipitation of solid particles.

### DETAILED DESCRIPTION OF THE INVENTION

This invention comprises personal care products made with this new type of silicone elastomer. The new elastomer is comprised of multiple units which may be the same or different for which each unit is a quaternary amino functional silicone elastomer of Formula I:

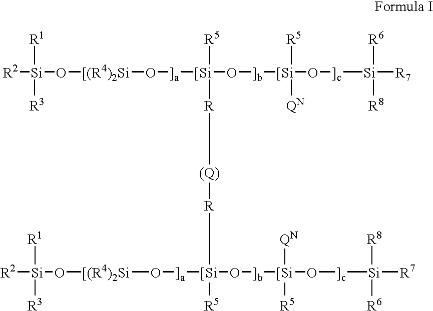

where:

R¹, R², R³, R⁴, R⁵, R⁶, R⁷ and R⁸ may be the same or different and are each independently selected from the group consisting of C1-C10 monovalent hydrocarbon radicals (preferably from the group consisting of methyl, ethyl, propyl, isopropyl, and n-butyl; and most preferably methyl);

R is a divalent hydrocarbon radical of formula —(CH₂)ₛ— where s is a number in the range of 2-10 (particularly 2-8) and R is preferably —CH₂—CH₂—;

a=is an average value and is a number in the range of 0-500 (particularly 10-150);

b=is an average value and is a number in the range of 1-150 (particularly 1-15);

c=is an average value and is a number in the range of 1-50 (particularly 1-5);

provided that b+c is a number in the range of 2-200 (particularly 2-30);

Q is a siloxane chain selected from the group consisting of Q^{1A} as shown in Formula VA and Q^{1B} as shown in Formula VB; and

Q^{N} is a quaternary amino polysiloxane pendent group having quaternary nitrogen groups and represented by Formula VIII.

The units of Formula I are crosslinked at structure Q to create an elastomer of the selected molecular weight. In particular, the average molecular weight of the material of Formula I should be in the range of 50,000-650,000, preferably 70,000-200,000, and especially 100,000 daltons. One source of quaternized elastomers of Formula I is Reliance Silicones (India) Limited.

One way of describing the elastomer is describing a particular method of making compounds of Formula I (also referred to herein as quaternized silicone elastomers). The starting material is an organohydrogen polysiloxane copolymer of Formula II (which may be obtained from a supplier such as either from Reliance Silicones (India) Limited or Petrarch Systems Silanes & Silicones (a subsidiary of United Chemical Technologies, Sugarland, Tex.) and also referred to herein as a methyl hydrogen polysiloxane copolymer (“MHPS copolymer”).

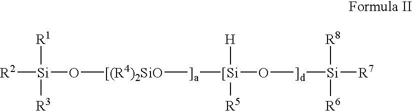

where:

R¹, R², R³, R⁴, R⁵, R⁶, R⁷ and R⁸ may be the same or different and are each independently selected from the group consisting of C1-C10 monovalent hydrocarbon radicals (particularly selected from the group consisting of methyl, ethyl, propyl, and isopropyl, and most preferably selected to be methyl groups);

d is a number equal to b+c (which is a number in the range of 2-200);

a, previously defined as a number in the range of 0-500.

More particular examples of copolymers of Formula II are those in which a and d are selected so that the material of Formula II has an average molecular weight in the range of 1,000-50,000.

The material of Formula II is partially reduced by using platinum-divinyl tetramethyldisiloxane complex in cyclomethicone (such as D₄ or D₅) or dimethicone (such as a 350 centistoke material) to partially convert the Si—H groups of Formula II material to Si—OH. This gives a material of Formula III.

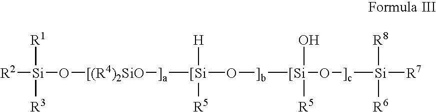

More cyclomethicone (particularly if an underarm product is being made later) or dimethicone (particularly if a hair care, skin cleanser or skin moisturizer is being made later) is added with at least one crosslinker selected from the group consisting of (a) an alkenyl functional gum of Formula IV:

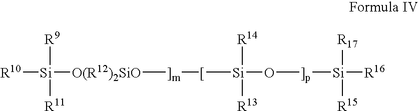

where,

m and p are selected so that the molecular weight of the material of Formula IV is in the range of 100,000-900,000, particularly 300,000-600,000;

R¹⁰, R¹³ and R¹⁶ may be the same or different and are each independently selected from the group consisting of C2-C10 monovalent hydrocarbon radicals having an unsaturation at the terminal carbon (that is, an alkyl or vinyl group, preferably vinyl);

R⁹, R¹¹, R¹², R¹⁴, R¹⁵ and R¹⁷ may be the same or different and are each independently selected from the group consisting of C1 to C10 monovalent hydrocarbon radicals (preferably, methyl, ethyl, propyl, isopropyl; most preferably methyl). Note that for R¹³ in Formula IV, additional crosslinking may take place at this reactive alkenyl site so that a three dimensional structure is created via the reaction of the terminal unsaturated bond of R¹³, however, the sites where R¹⁰ and R¹⁶ are located will react first. Materials of Formula IV may also be obtained from Petrarch Systems Silanes & Silicones.

(b) an alkenyl terminated polysiloxane of Formula V:

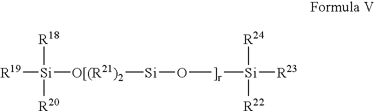

where:

r is an average number selected to give the material of Formula V an average viscosity in the range of 1,000-100,000 centistokes;

R¹⁹, R²³ may be the same or different and are each independently selected from the group consisting of C2-C10 monovalent unsaturated hydrocarbon radicals having 2 to 10 carbon atoms with an unsaturation at the terminal carbon (that is an allyl or vinyl group, preferably vinyl); and

R¹⁸, R²⁰, R²¹, R²² and R²⁴ may be the same or different and are each independently selected from the group consisting of C1 to C10 monovalent hydrocarbon radicals (preferably selected from the group consisting of methyl, ethyl, propyl, isopropyl, and most preferably selected to be methyl) (Materials of Formula V may also be obtained from Petrarch Systems Silanes & Silicones or Reliance Silicones.); and

(c) mixtures of (a) and (b) particularly where the ratio of (a): (b) is in the range of 1:20-1:50, and more particularly with a ratio of 1:30.

In these definitions the use of a cross linker of Formula IV will give Q^{1A} the following structure:

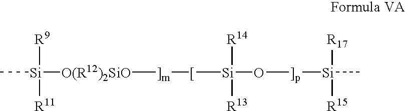

and the use of a crosslinker of Formula V will give Q^{1B} the following structure:

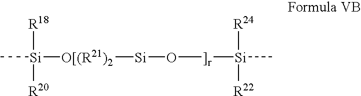

The vinyl-terminated polysiloxane of Formula V can be obtained from Reliance Silicones (India) Limited or Petrarch Systems Silanes & Silicones.

The mixture of the material of Formula III with the crosslinker (one or more crosslinkers) is homogenized with heating under conventional conditions in the presence of cyclomethicone (or dimethicone) and a platinum catalyst, until the mixture gels. This gelled material, in general, may be represented by Formula VI:

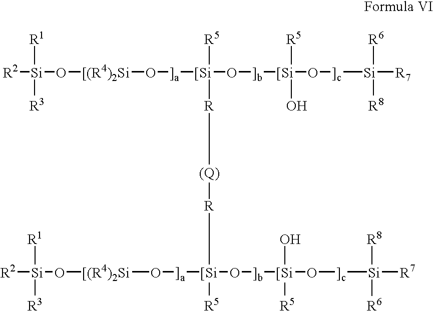

with preferred values for R¹, R², R³, R⁴, R⁵, R⁶, R⁷ and R⁸ being methyl; and values for R and Q being the same as described in Formula I.

To make the quaternary product more cyclomethicone (or dimethicone) is added with homogenization and further reaction is carried out with a reactive quaternary silicone such as a dialkoxy (preferably dimethoxy or diethoxy) terminated quaternary amino silicone of Formula VII:

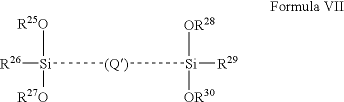

where:

R²⁵, R²⁶, R²⁷, R²⁸, R²⁹ and R³⁰ may be the same or different and are each independently selected from the group consisting of C1-C10 monovalent hydrocarbon radicals (preferably from the group consisting of methyl, ethyl, propyl, isopropyl; most preferably methyl);

Q′ is a quaternary amino functional polydimethylsiloxane such as

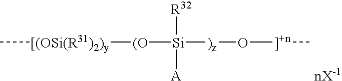

where:

A=a di-cation of formula —(CH₂)₃N(R³⁷)₂⁺CH₂CH₂N(R³⁸)₃⁺¹ or a mono-cation of formula —(CH₂)₃N(R³⁹)₃⁺¹; X⁻¹ is selected from the group consisting of CH₃COO⁻¹; (SO₄)⁻²; (SO₃)⁻¹; Cl⁻¹; I⁻¹; and F⁻¹ (particularly CH₃COO⁻¹; I⁻¹ and Cl⁻¹);

R³¹ and R³² may be the same or different and are each selected from the group consisting of C1-C10 monovalent hydrocarbon radicals (preferably selected from the group consisting of methyl, ethyl, propyl, and isopropyl; and most preferably selected to be methyl); R³⁷, R³⁸ and R³⁹ may be alike or different and are each independently selected from the group consisting of hydrogen, C1-C10 monovalent hydrocarbon radicals (preferably selected from the group consisting of methyl, ethyl, propyl and isopropyl; and most preferably methyl);

y is an average value and is a number in the range of 1-750; and

z is an average value and is a number in the range of 1-200.

The dimethoxy terminated quaternary silicones of Formula VII can be made by quaternizing dimethoxy terminated amino silicone (such as dimethoxy terminated polydimethyl aminoethyl aminopropyl methylsiloxane from Shin-Etsu Silicones of America, Akron, Ohio) using acetic acid, HI, HCl or an alkyl halide.

The material of Formula VII reacts at its alkoxy sites (for example, methoxy sites) with the Si—OH groups of Formula VI to give the material of Formula I. In the process of doing this, a pendent group of Formula VIII is formed:

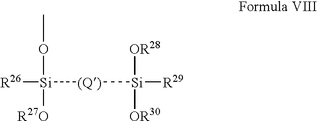

which leads to the formation of the quaternary amino functional silicone elastomer of Formula I described above. (Formula VIII is the same as Q^{N}). The elastomer is insoluble in volatile silicone, organic and inorganic solvents.

The amino polysiloxanes are quaternized using protonic acids (such as HCl, HI, acetic acid) or alkyl halides give quaternary amino polysiloxanes. For each of these example amines the counter ion X⁻¹ may be selected from the group consisting of CH₃ COO⁻¹, I⁻¹ or Cl⁻¹.

Other options for quaternary functional silicones include, (1) trialkoxy functional quaternary amino silicone (preferably trimethoxy or triethoxy); and (2) monoalkoxy functional quaternary amino silicone (preferably monomethoxy or monoethoxy). Also mono, di and tri alkoxy functional amino functional silicones may be used separately or along with quaternary functional silicones. Alkoxy or amino functionality may be present at terminal and/or within the polysiloxane chain. All these above functional silicones may be linear and or branched.

Suitable catalysts for the partial reduction step include Group VIII transition metals, preferably platinum metal, rhodium metal or their respective complexes. Such catalysts can be used separately or in a mixture.

Silicone fluids used in the preparation of the quaternized elastomer paste include low molecular weight linear and/or cyclic volatile silicone fluids; and branched chain volatile and non-volatile silicone fluids. In more specific terms,

(a) linear volatile silicone fluids such as hexamethyldisiloxane (MM), octamethyltrisiloxane, decamethyltetrasiloxane, dodecamethylpentasiloxane, tetradecamethylhexasiloxane, hexadecamethylheptasiloxane, etc.

(b) cyclic volatile silicones of the general formula ((R³³)₂SiO)ᵢ (where i<8) (particularly hexamethylcyclotrisiloxane (D₃), octamethylcyclotetrasiloxane (D₄), decamethylcyclopentasiloxane (D₅), dodecamethylcyclohexasiloxane (D₆))

(c) branched volatile methyl siloxanes heptamethyl-3-{(trimethylsilyl)oxy}trisiloxane (M₃T); hexamethyl-3,3,bis{(trimethylsilyl)oxy} trisiloxane (M₄Q), pentamethyl {(trimethylsilyl)oxy}cyclotrisiloxane (MD₃).

(d) cyclic non-volatile silicones of the general formula ((R³³)₂SiO)ⱼ (where j≧8).

(e) linear non volatile polysiloxanes of the formula (R³⁴)₃SiO((R³⁵)₂SiO)ₕSi(R³⁶)₃ having viscosity in the range of 5-2000 cps, where R³³, R³⁴, R³⁵ and R³⁶ may be the same or different and are each selected from the group consisting of C1-C10 monovalent hydrocarbon radicals (preferably selected from the group consisting of methyl, ethyl, propyl, and isopropyl; and most preferably selected to be methyl) or aryl; and h defined as a number in the range of 2-500, preferably in the range of 10-250; more preferably 10-100.

Other than silicone fluids, organic compounds (mainly solvents) can also be used to prepare the elastomer paste either separately or in the mixture form (along with silicone fluids). These include organic solvents such as alcohols (for example methanol, ethanol, 1-propanol, cyclohexanol, benzyl alcohol, 2-octanol, ethylene glycol, propylene glycol, and glycerol); aromatic hydrocarbons (for example benzene, toluene, ethylbenzene, and xylene); aliphatic hydrocarbons (for example cyclohexane, heptane); aldehydes, (for example acetaldehyde, benzaldehyde); ketones (for example acetone, methyl ethyl ketone, cyclohexanone, diacetone alcohol, methyl amyl ketone, and diisobutyl ketone); amines (for example isopropylamine, cyclohexylamine, ethanolamine, and diethanolamine); esters (for example ethyl acetate, isopropyl acetate, ethyl acetoacetate, amyl acetate, isobutyl isobutyrate, and benzyl acetate); ethers (for example ethyl ether, n-butyl ether, tetrahydrofuran, and 1,4-dioxane; glycol ethers); glycol ethers (for example ethylene glycol monomethyl ether, ethylene glycol monomethyl ether acetate, diethylene glycol monobutyl ether, and propylene glycol monophenyl ether); alkyl halides (for example chloroform, carbon tetrachloride, chlorobenzene); petroleum hydrocarbons (for example mineral oil, kerosene); gas oil, heavy oil, crude oil, lubricating oil; naturally occurring fatty oil (for example corn oil, soybean oil, olive oil, rape seed oil, cotton seed oil, sardine oil, herring oil, and whale oil).

The invention includes products such as antiperspirants and/or deodorants having a formula made with at least 10 weight % of a volatile silicone. For example, if a stick is desired, a gellant such as a minimum of 10 weight % stearyl alcohol or a minimum of at least 5 weight % siliconized polyamide as described in U.S. Pat. Nos. 5,919,441 and 6,051,216 (incorporated herein by reference as to their description of gelling agents) may be used. If a gel is desired, a surfactant in the range of 0.1-3 weight % is added.

If a gelling agent is used such such agents also include those described in U.S. Pat. Nos. 5,833,964; 5,730,963; 5,635,164; and 5,462,736, all of which are incorporated herein by reference as to their description of solidifying agents.

Examples of antiperspirants and/or deodorants include:

Gel Made with

Oil Phase:

17.9-69.7 weight % cyclomethicone (preferably D5)

0.1-25 weight % quaternized silicone elastomer of Formula I

2-15 weight % silicone copolyol (DC 5225C from Dow Corning Corp.)

0-20 weight % emollient

0-5 weight % fragrance

Aqueous Phase:

0.1-25 weight % antiperspirant active (such as Rezal 36 GP from Reheis Corp. Berkeley Heights, N.J.; or Zr 35BX from Westwood Chemical Corp., Middletown, N.J.) 20-79.9 weight % water

0-10 weight % of one or more of ethanol, propylene glycol, dipropylene glycol or tripropylene glycol

0-10 weight % of an organic or inorganic salt (such as sodium chloride, zinc chloride or magnesium chloride)

Stick A Made with

Part 1:

10-21 weight % cyclomethicone (such as DC 345 from Dow Corning Corp, Midland Mich.)

0-6 weight % PPG-3 myristyl ether

6-11 weight % C12-15 alkyl benzoate (such as Finsolv TN from Finetex Inc., Elmwood Park, N.J.)

18-25 weight % stearyl alcohol (from Cognis, Ambler, Pa.)

0-10 weight % (especially 4%) hydrogenated castor oil (such as MP 80 from Caschem, Bayonne, N.J.)

0-10 weight % (especially 4%) (PEG-8 distearate (from Stepan Chemicals, Northfield, Ill.)

up to 10.0 weight % quaternized elastomer of Formula I

Part 2:

15-25 weight % (especially 20%) cyclomethicone as above

0-15 weight % (especially 5%) dimethicone copolyol (DC 5225C from Dow Corning,

10% active in cyclomethicone)

17-25 weight % antiperspirant active (such as Reach AZP 908 from Reheis Corp.)

0-5 weight % fragrance

Soft Solid

5-70 weight % quaternized silicone elastomer of Formula I

0.1-25 weight % antiperspirant active

0-20 weight % fumed silica

0-5 weight % fragrance

0-10 weight % dimethicone copolyol (DC 5225C)

QS Cyclomethicone

For antiperspirants and deodorants the ingredients can be chosen from the following.

The antiperspirant active can be selected from the group consisting of any of the known antiperspirant active materials. These include, by way of example (and not of a limiting nature), aluminum chlorohydrate, aluminum chloride, aluminum sesquichlorohydrate, zirconyl hydroxychloride, aluminum-zirconium glycine complex (for example, aluminum zirconium trichlorohydrex gly, aluminum zirconium pentachlorohydrex gly, aluminum zirconium tetrachlorohydrex gly and aluminum zirconium octochlorohydrex gly), aluminum chlorohydrex PG, aluminum chlorohydrex PEG, aluminum dichlorohydrex PG, and aluminum dichlorohydrex PEG. The aluminum-containing materials can be commonly referred to as antiperspirant active aluminum salts. Generally, the foregoing metal antiperspirant active materials are antiperspirant active metal salts. In the embodiments which are antiperspirant compositions according to the present invention, such compositions need not include aluminum-containing metal salts, and can include other antiperspirant active materials, including other antiperspirant active metal salts. Generally, Category I active antiperspirant ingredients listed in the Food and Drug Administration's Monograph on antiperspirant drugs for over-the-counter human use can be used. In addition, any new drug, not listed in the Monograph, such as aluminum nitratohydrate and its combination with zirconyl hydroxychlorides and nitrides, or aluminum-stannous chlorohydrates, can be incorporated as an antiperspirant active ingredient in antiperspirant compositions according to the present invention.

Particular types of antiperspirant actives include aluminum zirconium trichlorohydrex and aluminum zirconium tetrachlorohydrex either with or without glycine. A particular antiperspirant active is aluminum trichlorohydrex gly such as AZZ-902 SUF (from Reheis Inc., Berkley Heights, N.J.) which has 98% of the particles less than 10 microns in size. Antiperspirant actives can be incorporated into compositions according to the present invention in amounts in the range of 0.1-25% of the final composition, but the amount used will depend on the formulation of the composition. For example, at amounts in the lower end of the broader range (for example, 0.1-10% on an actives basis), a deodorant effect may be observed. At lower levels the antiperspirant active material will not substantially reduce the flow of perspiration, but will reduce malodor, for example, by acting as an antimicrobial material. At amounts of 10-25% (on an actives basis) such as 15-25%, by weight, of the total weight of the composition, an antiperspirant effect may be observed.

Emollients are a known class of materials in this art, imparting a soothing effect to the skin. These are ingredients which help to maintain the soft, smooth, and pliable appearance of the skin. Emollients are also known to reduce whitening on the skin and/or improve aesthetics. Examples of chemical classes from which suitable emollients can be found include:

(a) fats and oils which are the glyceryl esters of fatty acids, or triglycerides, normally found in animal and plant tissues, including those which have been hydrogenated to reduce or eliminate unsaturation. Also included are synthetically prepared esters of glycerin and fatty acids. Isolated and purified fatty acids can be esterified with glycerin to yield mono-, di-, and triglycerides. These are relatively pure fats which differ only slightly from the fats and oils found in nature. The general structure may be represented by Formula VI:

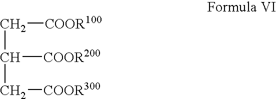

wherein each of R¹⁰⁰, R²⁰⁰, and R³⁰⁰ may be the same or different and have a carbon chain length (saturated or unsaturated) of 7 to 30. Specific examples include peanut oil, sesame oil, avocado oil, coconut, cocoa butter, almond oil, safflower oil, corn oil, cotton seed oil, castor oil, hydrogenated castor oil, olive oil, jojoba oil, cod liver oil, palm oil, soybean oil, wheat germ oil, linseed oil, and sunflower seed oil.

(b) hydrocarbons which are a group of compounds containing only carbon and hydrogen. These are derived from petrochemicals. Their structures can vary widely and include aliphatic, alicyclic and aromatic compounds. Specific examples include paraffin, petrolatum, hydrogenated polyisobutene, and mineral oil.

(c) saturated and unsaturated fatty alcohols (primary, secondary and tertiary alcohols, and including guerbet alcohols) with general structure:

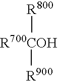

wherein each of R⁷⁰⁰, R⁸⁰⁰ and R⁹⁰⁰ is hydrogen or a straight or branched chain carbon group and the total number of carbons in R⁷+R⁸+R⁹ is in the range of 7-30. Specific examples include lauryl, myristyl, cetyl, isocetyl, stearyl, isostearyl, oleyl, ricinoleyl and erucyl alcohol.

(d) lanolin and its derivatives which are a complex esterified mixture of high molecular weight esters of (hydroxylated) fatty acids with aliphatic and alicyclic alcohols and sterols as well as propoxylated and/or butoxylated species. Specific examples include lanolin, lanolin oil, lanolin wax, lanolin alcohols, lanolin fatty acids, isopropyl lanolate, propoxylated lanolin, butoxylated lanolin, and acetylated lanolin alcohols.

(e) alkoxylated alcohols wherein the alcohol portion is selected from aliphatic alcohols having 2-18 and more particularly 4-18 carbons, and the alkylene oxide portion is selected from the group consisting of propylene oxide and butylene oxide having a number of alkylene oxide units from 2-53 and, more particularly, from 2-15. Specific examples include PPG-14 butyl ether and PPG-53 butyl ether.

(f) miscellaneous which are selected from the group consisting of neopentyl glycol diheptanoate, PEG-8 laurate, isocetyl stearate, dimethicone copolyol laurate, Dow Corning 2501 cosmetic wax (dimethicone copolyol); isostearyl isostearate, isostearyl palmitate, isostearyl alcohol, PPG-5-ceteth-20, PPG-10-cetyl ether, triethyl hexanoin, ethyl hexyl isostearate, glyceryl oleate, and isopropyl isostearate.

(g) mixtures and blends of two or more of the foregoing.

Particular examples of suitable emollients include members of the group consisting of Octyloxyglyderin (SENSIVA SC50 from Schülke Mayr, Norderstedt, Germany) (which can be used as an emollient as well as an antibacterial); Polysorbate 80 (TWEEN 80 from ICI Americas, Wilmington, Del.); Oleth-20;

ethoxylated alcohols such as steareth-2, nonoxynol-2, PPG-4-Ceteth-1; ethoxylated carboxylic acids such as PEG-4 dilaurate, PEG-2 oleate; glyceryl esters such as PEG-2 castor oil, polyglyceryl-3 oleate, glyceryl stearate; sorbitan derivatives such as sorbitan oleate; PPG-3 myristyl ether (such as WITCONOL APM from Goldschmidt), a dimethiconol (such as Dow Corning® DC1501 dimethiconol), neopentyl glycol diheptanoate, PEG-8 laurate, isocetyl stearate, dimethicone copolyol laurate, Dow Corning 2501 cosmetic wax (dimethicone copolyol); isostearyl isostearate, isostearyl palmitate, isostearyl alcohol, PPG-5-ceteth-20, PPG-10-cetyl ether, triethyl hexanoin, ethyl hexyl isostearate, glyceryl oleate, and isopropyl isostearate.

The silicone materials used in forming the compositions of the present invention may be selected from the group consisting of conventional cyclic and linear volatile and non-volatile silicones which act as a swelling agent for the suitable elastomer. Illustratively, and not by way of limitation, the volatile silicones are one or more members selected from the group consisting of cyclic polydimethylsiloxanes such as those represented by Formula IB:

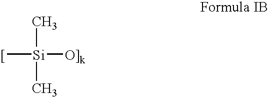

where k is an integer with a value of 3-7, particularly 5-6. By volatile silicone material is meant a material that has a measurable vapor pressure at ambient temperature. For example, DC-245 fluid from Dow Corning Corporation (Midland, Mich.) is a type of cyclomethicone which can be used. These include a tetramer (or octylmethylcyclotetrasiloxane) and a pentamer (or decamethylcyclopentasiloxane). The nonvolatile and volatile linear silicones are one or more members selected from the group consisting of linear polydimethylsiloxanes such as those represented by Formula IIB:

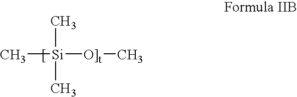

and t is selected so that the viscosity ranges from 5-600,000 centistokes (for example Dimethicone DC 200 from Dow Corning).

Other types of personal care products can also be made. Some examples include the following.

Hair Conditioner

Part 1:

0.10-0.80 weight % hydroxyethyl cellulose (such as Number 2 from Hercules, Wilmington, Del.) (particularly 0.3%)

0.20-2.00 weight % (particularly 0.8%) cetyl trimethyl ammonium chloride (25% active)

QS water (especially deionized) (particularly 50%)

Part 2:

0.50-3.00 weight % behenyl alcohol (particularly 1.3%)

0.10-1.00 weight % distearyl dimmonium chloride DSDAC (particularly 0.6%)

0.10-2.00 weight % stearyl alcohol (particularly 0.5%)

0.10-2.00 weight % cetyl alcohol (particularly 0.5%)

0.25-2.00 weight % mineral oil (particularly 1.0%)

Part 3:

0.10-2.00 weight % (particularly 1.0%) quaternized silicone elastomer of Formula I in dimethicone (14% active)

Part 4:

0-1.00 weight % fragrance (particularly 0.2%)

0.05-0.40 formalin (particularly 0.1%)

Shampoo

0.05-0.80 weight % tetrasodium EDTA

10.0-40.0 weight % sodium lauryl ether sulfate (25.5% active)

2.00-12.00 weight % betaine

0.50-4.00 weight % of a cationic conditioning agent (such as Polyquaternium-7)

0.10-2.00 weight % quaternized silicone elastomer of Formula I in dimethicone (14% active)

1.00-8.00 behenyl alcohol dispersion (25% active)

0-2.00 weight % fragrance

0.05-0.40 weight % preservative (such as DMDM hydantoin)

QS water (especially deionized)

Mousse

Part 1:

3.00-10.00 weight % ethanol (SD 40-B)

QS water (especially deionized)

Part 2:

70-90 weight % water (especially deionized) (especially 3.00%)

0.20-0.50 weight % Polyquaternium-10 (Polymer JR)

Part 3:

1.00-3.00 weight % amphomer (octylacrylamide/acrylates/butylaminoethylmethacrylate copolymer)

0-0.50 weight % fragrance)

0.30-1.00 weight % propylene glycol

0.20-2.00 weight % quaternized silicone elastomer of Formula I

0.20-1.00 weight % cocodiethanolamide

Part 4:

3.00-8.00 weight % hydrocarbon propellant (such as A-46)

0.20-0.60 weight % preservative (such as DMDM hydantoin)

Body Wash

Part 1:

QS water (such as deionized)

0.05-0.80 weight % tetrasodium EDTA (particularly 0.2%)

Part 2:

30-70 weight % water (such as deionized) (particularly 3%)

0.50-4.00 weight % cationic conditioning agent (such as Polyquaternium-7) (particularly 3%)

Part 3:

10.00-40.00 weight % (particularly 39.216%) sodium lauryl ether sulfate (25.5% active)

2.00-12.00 weight % betaine (such as C10-24 alkylamidopropylbetaine) (particularly 10%)

Part 4:

0.50-4.00 weight % decyl glucoside (particularly 2%)

0-2.00 weight % fragrance (particularly 1%)

Part 5:

0.10-2.00 weight % quaternized silicone elastomer of Formula I (particularly 1.0%)

0.05-0.40 weight % preservative (such as DMDM hydantoin) (particularly 0.40%)

### EXAMPLES

The following Examples are offered as illustrative of the invention and are not to be construed as limitations thereon. In the Examples and elsewhere in the description of the invention, chemical symbols and terminology have their usual and customary meanings. In the Examples as elsewhere in this application values for n, m, etc. in formulas, molecular weights are averages. For Formula I material and corresponding starting materials, alkyl groups of the various R designations are methyl. Temperatures are in degrees C. unless otherwise indicated. The amounts of the components are in weight percents based on the standard described; if no other standard is described then the total weight of the composition is to be inferred. Various names of chemical components include those listed in the CTFA International Cosmetic Ingredient Dictionary (Cosmetics, Toiletry and Fragrance Association, Inc., 7^{th} ed. 1997). Unless otherwise indicated in this application, viscosity may be measured by using Brookfield viscometer (LV or RVmodels) at 25 degree C. (for example, when the viscosity is above 100,000 cps, E-spindle is used, and the speed is set at 2.5 rpm; when the viscosity is between 10,000-100,000 cps, spindle #4 is used at a rotation setting of 2.5 rpm); Other appropriate spindles for the ranges of viscosities being measured may also be used, such as a # 4 spindle and 30 rpm. Additionally, some vendors specify the techniques for measuring viscosity in their trade literature. Materials with centistoke data (“cst”) reflect data obtained from vendors. These data reflect the methods described by those vendors.

### Example 1

### Preparation of an Antiperspirant Product

The elastomer paste of the type described above may be mixed with D₅ cyclomethicone (DC 245 from Dow Corning Corp, Midland, Mich.), fragrance, PPG-3 myristyl ether (Varonic APM from Goldschmidt Chemical Corporation, Hopewell, Va.) and a silicone emulsifier (DC 5225C from Dow Corning Corp., Midland, Mich.). The mixture is stirred at about 500 rpm using an overhead mixer such as a Lightnin Mixer Model L1003 for 15-20 minutes or until it is visually homogeneous. Water, antiperspirant active, and other water soluble ingredients which are premixed in a separate beaker are then added gradually to the silicone mixture with continuous stirring. Stirring is maintained for another 20 minutes after the addition is complete. The mixture is then homogenized for 2 minutes to achieve the desired viscosity (50,000 cps -300,000 cps) using a Gilford-Wood Model 1-L.(Greerco Corp., Hudson, N.H.) homogenizer at a reading of 60 on a Powerstat Variable Autotransformer from Superior Electric Co., Bristol, Conn.

### Example 2

### Preparation of Quaternized Elastomer with Other Silicones

For hair care and body cleansing and/or moisturizing, it is desirable to use other types of silicones rather than a cyclomethicone.

An MHPS copolymer (25 g) (for example, the MHPS copolymer described in for Formula II) and dimethicone having a general formula (R³⁴)₃SiO((R³⁵)₂SiO)ₕSi(R³⁶)₃ and having a viscosity of 350 cps described above (DM 350 from Reliance Silicones) are placed in a 500 milliliter beaker. A 10% solution of platinum - divinyl tetramethyldisiloxane complex in cyclomethicone, preferably D₅ or D₄ (0.25 g) is added. The reaction mixture is stirred for 10 minutes at 100 rpm. A vinyl terminated polysiloxane (15 g) (for example, a vinyl terminated polysiloxane as described in Formula V) and a 10% solution of vinyl gum solution in cyclomethicone (5 g) (for example, a vinyl gum of Formula IV) are also added with stirring. The mixture is heated to 40-45 degrees C. More of the platinum - divinyl tetramethyldisiloxane complex solution described above is added dropwise (1.5 g) and the mixture is stirred until it gels. Next more dimethicone is added (100 g) and the mixture is homogenized at 2000-4000 rpm using a homogenizer (Remi Motors, Mumbai, India) until a thick paste is obtained. The temperature of elastomer paste is maintained between 25-30 degree C. The elastomer paste is further diluted and homogenized with 70 g of dimethicone. Next, dimethoxy-terminated quaternary amino fluid (15 g) (for example, a quaternary derivative of dimethoxy terminated aminofunctional polysiloxane as described in Formula VII) and dimethicone (35 ml) are added to the elastomer paste and homogenization is continued until a soft paste is obtained. The amount of quaternized elastomer obtained is 367 g with a dimethicone content of 311 g (84.74 weight %). The elastomer content is 56 g (15.26 weight %).

### Example 3

### Preparation of 2-in-1 Shampoo

A hair care product such as a 2-in-i shampoo may be made by using a quaternized elastomer of Formula I with dimethicone, especially one having a viscosity of 350-1000 centistokes. To the main mixing vessel, water is mixed with sodium lauryl ether (2EO) sulfate (Colgate-Palmolive) for 15 minutes or until uniform. Then, to the main mixing vessel, cocoamidopropyl betaine (Tego Betaine L7, from Goldschmidt in Hopewell, Va.) is gradually added and mixed for 15 minutes or until uniform. Once the batch is clear and uniform, Polyquatemium -7 (Merquat 550, from Calgon, in Ill.) is slowly added and mixed until uniform. Then to the main mixing vessel, behenyl alcohol pearl (Lamesoft K E, from Cognis in Germany) is added to achieve a stable emulsion. After the batch is uniform, the quaternized elastomer (15.35% elastomer active in 84.65% of 350 cst dimethyl polysiloxane) (Reliance Silicones F15, Maharashtra, India) is added and mixed for 15 minutes. Upon uniformity of the batch being achieved, appropriate amounts of preservatives and colors are sequentially added. Next, viscosity and pH modifiers are added to achieve the desired viscosity (5000-7000 cps) using a Brookfield RVTP, spindle #4, 20 RPM at 25° C. for 60 seconds and VWR model 8000.

### Example 4

### Preparation of 2-in-1 Shampoo

A shampoo of a 100 gram batch size can be made with the ingredients as listed in percent by weight based on the entire composition: 0.25% sodium EDTA (40% solution); 40% sodium pareth C12-13 sulfate (25% active); 7.5% cocamidopropyl betaine (30% solution);2.81% Polyquaternium-7 (8% solution); 0.50% quaternized silicone elastomer of Formula I (with alkyl R groups as methyl) (14% active in dimethicone); 4.0% behenyl alcohol dispersion (25% active); 0.45% fragrance; 1.698% coloring agents (0.1-1% in solution); 0.40% DMDM hydantoin; 6.50% NaCl (20% solution); ±0.10% citric acid (50% solution); QS filtered irradiated demineralized water. A major portion of water is added to a main mixing vessel (stainless steel type 304L or 316L) equipped with a variable speed Lightnin' Mixer and heating and cooling capabilities. Mixing is begun. The sodium EDTA is added and mixed for at least 5 minutes or until uniform. The sodium pareth C12-13, 2 EO sulfate is added with care not to promote foam formation. Mixing is continued for at least 15 minutes or until uniform. The CAP betaine is then added and mixing is continued for 20 minutes or until uniform. The Polyquaternium-7 is then added and mixing is continued for 15 minutes or until uniform. The quaternized silicone is added and mixing is continued for at least 20 minutes or until uniform. The behenyl alcohol dispersion is added and mixing is continued for at least 20 minutes or until uniform. The fragrance is added and mixing is continued for at least 15 minutes or until uniform. The citric acid is added if needed to lower pH (target=6.25) and mixing is continued for 5 minutes. The coloring agents are added and mixing is continued for 5 minutes. The NaCl solution is added to adjust the viscosity of the product by thickening it (target=6000 cps). The products will be thick and a sweep mixer will be needed to mix. The batch is mixed for 20 minutes. Adjust temperature to about 25 degrees C. Batch may be discharged through a 20 mesh (or equivalent) filter when below 30 degrees C.

### Example 5

### Preparation of Bar Soap

A bar soap product may be made with the following ingredients: 50-99.9% soap chips (85/15 tallow/coconut) 0.1-50% quaternized silicone elastomer in dimethicone (14% active); and 0-5% fragrance. The soap chips (which themselves may also contain up to 10% water) are mixed with the other ingredients in an amalgamator. The chips are twice milled, plodded and pressed into soap bars. All %'s are weight % based on the total weight of the composition.

We claim:

1. A personal care product selected from the group consisting of antiperspirants and/or deodorants, soaps, and hair care products and made with a quaternary amino functional silicone elastomer having a molecular weight in the range of 50,000-650,000 daltons and comprising units which may be the same or different wherein each unit is represented by Formula I: 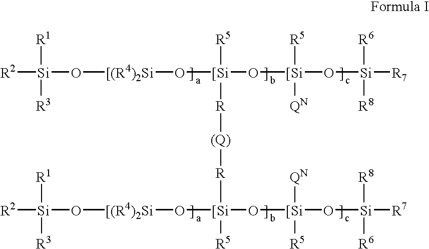
where:
R¹, R², R³, R⁴, R⁵, R⁶, Rand R⁸ may be the same or different and are each independently selected from the group consisting of C1-C10 monovalent hydrocarbon radicals;
R is a divalent hydrocarbon radical of formula —(CH₂)ₛ— where s is a number in the range of 2-10;
a=is an average value and is a number in the range of 0-500;
b=is an average value and is a number in the range of 1-150;
c=is an average value and is a number in the range of 1-50;
provided that b+c is a number in the range of 2-200;
Q is a siloxane chain selected from the group consisting of Q^{1A} of Formula VA: 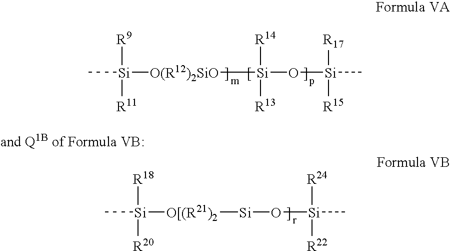
where
m is an average number with a value in the range of 2,000-10,000;
p is an average number with a value in the range of 1-200;
R¹³ is selected from the group consisting of C₂-C₁₀ monovalent hydrocarbon radicals having an unsaturation at the terminal carbon which may be used as a crosslinking site at the unsaturation;
R⁹, R¹¹, R¹², R¹⁴, R¹⁵ and R¹⁷ may be the same or different and are each independently selected from the group consisting of C1 to C10 monovalent hydrocarbon radicals;
R¹⁸, R²⁰, R²¹, R²² and R²⁴ may be the same or different and are each independently selected from the group consisting of C1 to C10 monovalent hydrocarbon radicals; and
Q^{N} is a quaternary amino polysiloxane pendent group of Formula VIII: 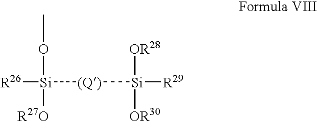
where
R²⁶, R²⁷, R²⁸, R²⁹ and R³⁰ may be the same or different and are each independently selected from the group consisting of C1-C10 monovalent hydrocarbon radicals; and
Q′ is a quaternary amino functional polydimethylsiloxane of formula: 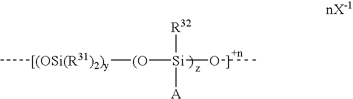
where:
A=a di-cation of formula —(CH₂)₃N(R³⁷)₂⁺CH₂CH₂N(R³⁸)₃⁺¹ or a mono-cation of formula —(CH₂)₃N(R³⁹)₃⁺¹; X⁻¹ is selected from the group consisting of CH₃COO⁻¹; (SO₄)⁻²; (SO₃)⁻¹; Cl⁻¹; I⁻¹; and F⁻¹ (particularly CH₃COO⁻¹; I⁻¹ and Cl⁻¹);
R³¹ and R³² may be the same or different and are each selected from the group consisting of C1-C10 monovalent hydrocarbon radicals;
R³⁷, R³⁸ and R³⁹ may be alike or different and are each independently selected from the group consisting of hydrogen, and C₁-C₁₀ monovalent hydrocarbon radicals.

2. A personal care product a claimed in claim 1 wherein the quaternary amino functional silicone elastomer of has a molecular weight in the range of 70,000-200,000 daltons.

3. A personal care product a claimed in claim 1 wherein the quaternary amino functional silicone elastomer has each of R¹, R², R³, R⁴, R⁵, R⁶, R⁷ and R⁸ as methyl.

4. A personal care product a claimed in claim 1 wherein the quaternary amino functional silicone elastomer has each of R⁹, R¹¹, R¹², R¹⁴, R¹⁵ and R¹⁷ as methyl.

5. A personal care product a claimed in claim 1 wherein the quaternary amino functional silicone elastomer has each of R¹⁸, R²¹, R²¹, R²² and R²⁴ as methyl.

6. A personal care product a claimed in claim 1 wherein the quaternary amino functional silicone elastomer has each of R²⁶, R²⁷, R²⁸, R²⁹ and R³⁰ as methyl.

7. A personal care product a claimed in claim 1 wherein the quaternary amino functional silicone elastomer has each of R³¹, R³², R³⁷, R³⁸ and R³⁹ as methyl.

8. A personal care product a claimed in claim 1 wherein the quaternary amino functional silicone elastomer has each of R¹, R², R³, R⁴, R⁵, R⁶, R⁷, R⁸, R⁹, R¹¹, R¹², R¹⁴, R¹⁵, R¹⁷, R¹⁸, R²⁰, R²¹, R²², R²⁴, R²⁶, R²⁷, R²⁸, R²⁹, R³⁰, R³¹, R³², R³⁷, R³⁸ and R³⁹ as methyl.

9. A personal care product a claimed in claim 1 which is an antiperspirant and/or deodorant comprising at least 10 weight % of a volatile silicone.

10. A personal care product a claimed in claim 1 which is a hair care product.

11. A personal care product a claimed in claim 1 which is a soap bar.

12. A personal care product a claimed in claim 1 which is a shower gel.

13. A personal care product a claimed in claim 1 which is a mousse.

14. A personal care product a claimed in claim 1 which is a shampoo.

15. A personal care product as claimed in claim 1 which is a hair conditioner.
